(19) United States
(12) Patent Application Publication
(10) Pub. No.: US 2026/0241115 A1
(43) Pub. Date: Aug. 20, 2026

(54) A sub-assembly of a medicament delivery device

(71) Applicant: SHL Medical AG, Zug (CH)

(72) Inventors: Caroline Wannehed Bergström, Nacka Strand (SE); Plamen Balkandjiev, Nacka Strand (SE); Daniel Carlsson, Nacka Strand (SE); Johan Zander, Nacka Strand (SE)

(21) Appl. No.: 19/489,630

(22) PCT Filed: Jun. 4, 2024
(86) PCT No.: PCT/EP2024/065343

§ 371 Date: Dec. 3, 2025

(30) Foreign Application Priority Data
Jun. 19, 2023 (EP) 23180163.0

(51) Int. Cl. A61M 5/46; A61M 5/20

(52) U.S. Cl. CPC A61M 5/46; A61M 5/20; A61M 2205/50; A61M 2205/587; A61M 2205/6081; A61M 2205/8206

(57) ABSTRACT

The present disclosure provides a sub-assembly of a medicament delivery device. The sub-assembly includes a housing extending along a longitudinal axis from a proximal end to a distal end. The sub-assembly further includes a processor accommodated within the housing and connected to one or more electronic components. The sub-assembly further includes a switch and a trigger each accommodated within the housing. The trigger comprises a sensing area. The switch comprises an electronic non-contact sensor attached to the housing and configured to sense the sensing area of the trigger. In response to sensing the sensing area, the switch is configured to provide a signal to the processor.

### TECHNICAL FIELD

The present disclosure generally relates to a sub-assembly of a medicament delivery device, and particularly to a sub-assembly of a medicament delivery device, the sub-assembly comprising a switch having an electronic non-contact sensor.

### BACKGROUND

Medicament delivery devices such as pen-type manual injectors or auto-injectors are generally known for the self-administration of a medicament by patients without formal medical training. For example, patients suffering from diabetes may require repeated injections of insulin, or patients may require regular injections of other types of medicaments, such as growth hormones.

It is an advantage when the medicament delivery devices for self-administration comprise automatic functions so that even if users are without professional training or are in an emergency situation, the users can easily and properly use the medicament delivery devices.

Even though many of the devices on the market, as well as the ones described above, have their respective advantages, there is still room for improvement.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a sub-assembly of a medicament delivery device comprising a medicament container, the sub-assembly comprising: a housing extending along a longitudinal axis from a proximal end to a distal end; wherein the housing is configured to be attached to a disposable cassette of the medicament delivery device; a processor connected to one or more electronic components; wherein the processor is accommodated within the housing; a switch accommodated within the housing; a trigger movably accommodated within the housing; wherein the trigger comprises a sensing area; wherein the trigger is configured to be move by a part of the disposable cassette. The switch comprises an electronic non-contact sensor attached to the housing and configured to sense the sensing area of the trigger. In response to sensing the sensing area, the switch is configured to provide a signal to the processor.

The trigger is configured to be moved by another component of the medicament delivery device within the housing such that the sensing area of the trigger can be moved relative to the electronic non-contact sensor.

As the traditional mechanical switch usually comprises multiple gaps between components, it usually cannot, or will be a complex design to provide a high ingress protection level. With an electronic non-contact sensor, a higher ingress protection level can be provided.

Preferably, according to another embodiment, the trigger is movable relative to the housing in the direction of the longitudinal axis.

Preferably, according to another embodiment, the trigger is rotatable relative to the housing around the longitudinal axis.

Preferably, according to another embodiment, the sub-assembly comprises a motor accommodated within the housing.

Preferably, according to another embodiment, the motor is configured to, upon being activated, cause expulsion of a medicament from the medicament container of the medicament delivery device.

Preferably, according to another embodiment, the processor, upon received the signal provided by the switch, is configured to activate the motor; or inactivate the motor; or pause the activation of the motor.

Preferably, according to another embodiment, the sub-assembly comprises a communication unit connected to the processor.

Preferably, according to another embodiment, the processor, upon received the signal provided by the switch, is configured to activate the communication unit.

Preferably, according to another embodiment, the housing comprises a distal section and a proximal section; wherein the switch is fully positioned within the distal section of the housing; and wherein the trigger is positioned within the proximal section of the housing.

Preferably, according to another embodiment, the proximal section of the housing comprises a channel configured to be releasably attached to a disposable cassette of the medicament delivery device.

Preferably, according to another embodiment, the medicament container is arranged within the disposable cassette; and wherein a portion of the trigger is positioned within the channel of the housing.

Preferably, according to another embodiment, the communication unit is positioned within the channel such that upon being activated, the communication unit is capable of reading and/or writing data from/to an information tag on the disposable cassette.

Preferably, according to another embodiment, the sensing area of the trigger comprises a sensing array having at least two sensing sections respectively lined up in a predetermined sequence.

Preferably, according to another embodiment, the electronic non-contact sensor is configured to sense different signals from the at least two sensing sections respectively.

Preferably, according to another embodiment, the sensing area of the trigger comprises a magnetic element.

Preferably, according to another embodiment, the sensing area of the trigger comprises a magnet.

Preferably, according to another embodiment, the sensing array of the trigger comprises a magnet array having at least two magnets with different polarities in the at least two sensing sections respectively.

Alternatively, according to another embodiment, the sensing area of the trigger is made of magnetic materials.

Preferably, according to another embodiment, the electronic non-contact sensor is a magnetic sensor.

Preferably, according to another embodiment, the switch is configured to provide the signal when the detected a predetermined magnetic field change.

Preferably, according to another embodiment, the predetermined magnetic field change can be at least one of the magnetic field strength exceeding a predetermined threshold, or a predetermined sequence of polarity changes.

Preferably, according to another embodiment, the electronic non-contact sensor comprises a Hall sensor.

Preferably, according to another embodiment, the electronic non-contact sensor comprises a reed switch.

Preferably, according to another embodiment, the electronic non-contact sensor comprises a magnetic meter.

Preferably, according to another embodiment, the electronic non-contact sensor comprises a magnet.

Preferably, according to another embodiment, the electronic non-contact sensor comprises a coil opening in the direction of the longitudinal axis. The sensor is configured to sense a current change on the coil.

Preferably, according to another embodiment, the electronic non-contact sensor is an optical sensor.

Preferably, according to another embodiment, the electronic non-contact sensor is a photoelectronic sensor, a color sensor, or a combination thereof.

Preferably, according to another embodiment, the at least two sensing sections are capable of providing different reflection signal respectively.

Preferably, according to another embodiment, the at least two sensing sections are coated with two different colors respectively.

Preferably, according to another embodiment, the at least two sensing sections are formed in two different structures respectively.

Preferably, according to another embodiment, the at least two sensing sections are a concave surface and a convex surface.

The above-mentioned sensing array allows more events of the medicament delivery device being sensed by the electronic non-contact sensor without increasing a number of the electronic non-contact sensors. As a result, more event controls can be provided by the processor without increasing the cost of the medicament delivery device. Furthermore, the sensing array also increases the accuracy of the detection as the target event is defined by both the presence of the sensing area and the presence of certain sections of the sensing area in a predetermined order. Thus, the impact from the environment noise can be reduced.

Preferably, according to another embodiment, the switch comprises two electronic non-contact sensors lined up to one another in the direction of the longitudinal axis.

Preferably, according to another embodiment, the switch is configured to provide at least two different signals to the processor based on different combinations of the detections from two electronic non-contact sensors respectively.

The two electronic non-contact sensors can be used to calibrate one another such that the detection accuracy can be increased. Furthermore, the two electronic non-contact sensors can also be used to detect the moving direction of the trigger, thus, more events of the medicament delivery device can be detected.

Preferably, according to another embodiment, the switch is a reed switch connected between a motor and a battery.

Preferably, according to another embodiment, the switch is a reed switch connected to the processor.

Preferably, according to another embodiment, the motor is configured to, upon being activated, output fluid towards the medicament to cause the expulsion of medicament from a medicament container of the medicament delivery device.

Preferably, according to another embodiment, the fluid is gas.

Preferably, according to another embodiment, the motor is a piezo micropump or an electrochemical engine.

Alternatively, according to another embodiment, the motor is connected to a lead screw such that upon the motor being activated, the motor moves the lead screw towards the medicament to cause the expulsion of medicament from a medicament container of the medicament delivery device.

Preferably, according to another embodiment, the motor comprises a rotor and a channel extending through the rotor. The lead screw is partially positioned within the channel.

Preferably, according to another embodiment, the channel comprises a thread extending around the channel. The thread is engaged with the thread of the lead screw.

Alternatively, according to another embodiment, the sub-assembly comprises a connector operably connected to the rotor such that the connector is configured to be rotated by the rotor.

Preferably, according to another embodiment, the connector comprises a channel extending through the connector and is aligned with the channel of the rotor along the longitudinal axis.

Preferably, according to another embodiment, the channel of the connector comprises a thread extending around the channel of the connector. The thread is engaged with the thread of the lead screw.

Preferably, according to another embodiment, the connector is attached to the rotor.

Preferably, according to another embodiment, the connector and the rotor are made of different materials.

Preferably, according to another embodiment, the connector is made of plastic.

Preferably, according to another embodiment, the rotor is made of metal.

Preferably, according to another embodiment, the longitudinal axis is the central axis of the motor.

Preferably, according to another embodiment, the motor is a stepper motor.

Preferably, according to another embodiment, the lead screw is rotationally immovable relative to the housing.

Preferably, according to another embodiment, an engagement formed between the lead screw and an inner wall of the housing is a ribs-ribs engagement or a ribs-grooves engagement.

Preferably, according to another embodiment, the motor is configured to reciprocate the movement of the lead screw.

Preferably, according to another embodiment, the trigger comprises a body having a distal section and a proximal section.

Preferably, according to another embodiment, the distal section comprises the sensing area.

Preferably, according to another embodiment, the proximal section comprises a first proximally directed surface and a second proximally directed surface extending from the first proximally directed surface towards a proximal end of the body.

Preferably, according to another embodiment, the body of the trigger comprises a distally directed surface; and the housing comprises a proximally directed surface. The sub-assembly comprises a biasing member positioned between the distally directed surface of the trigger and the proximally directed surface of the housing such that the trigger is biased towards the proximal end of the housing.

Preferably, according to another embodiment, a seal is positioned between the distal section of the housing and the proximal section of the housing.

The seal is configured to prevent the interior of the housing from water ingress.

Preferably, according to another embodiment, the seal is made of thermoplastic elastomers (TPE).

Preferably, according to another embodiment, the switch is fully positioned within the distal section of the housing.

Preferably, according to another embodiment, the trigger is positioned within the proximal section of the housing.

Preferably, according to another embodiment, an opening is arranged in a wall of the seal; wherein the distal section of the trigger comprises the sensing area; and wherein the distal section of the trigger is configured to be moved through the opening when the trigger is moved towards the distal end of the housing.

Preferably, according to another embodiment, the biasing member is positioned between the distally directed surface of the trigger and a proximally directed surface of the seal.

Preferably, according to another embodiment, the proximal section of the housing comprises a channel configured to be releasably attached to a disposable cassette of the medicament delivery device

Preferably, according to another embodiment, the medicament container is arranged within the disposable cassette.

Preferably, according to another embodiment, a portion of the trigger is positioned within the channel of the housing.

Preferably, according to another embodiment, the proximal section of the body of the trigger is positioned within the channel of the housing such that a component of the disposable cassette is capable of being in contact with the proximal section of the body of the trigger and moving the trigger towards the distal end of the housing.

Preferably, according to another embodiment, the sub-assembly comprises a battery connected to the processor.

Preferably, according to another embodiment, the sub-assembly comprises an indication unit connected to the processor.

Preferably, according to another embodiment, the information tag comprises at least one of RFID/NFC circuit, a barcode, a Quick Response code (QR code), and an inkjet label.

Preferably, according to another embodiment, the communication unit is configured to receive information from the disposable cassette.

Preferably, according to another embodiment, the communication unit is configured to write information to the information tag of the disposable cassette.

Preferably, according to another embodiment, the communication unit is configured to receive information about the medicament contained within the disposable cassette, a delivery dose of the medicament, and/or delivery sequence of the medicament from the disposable cassette.

Preferably, according to another embodiment, the communication unit is configured to write information about a residual volume of medicament within the disposable cassette and/or the material of components of the disposable cassette to the information tag of the disposable cassette.

Preferably, according to another embodiment, the communication unit is configured to be activated by the processor when the signal is sent by the switch.

Preferably, according to another embodiment, the signal is configured to be sent when a distal part of the cassette is in contact with the trigger and moves the trigger in the distal direction relative to the housing such that the sensing area of the trigger is sensed by the electronic non-contact sensor of the switch.

Preferably, according to another embodiment, a medicament delivery device comprises the sub-assembly as mentioned above; and a medicament container accommodated within the housing.

Alternatively, according to another embodiment, a medicament delivery device comprises a reusable drive unit comprising the sub-assembly as mentioned above.

Preferably, according to another embodiment, the medicament delivery device comprises a disposable cassette comprising a medicament container containing a medicament.

Preferably, according to another embodiment, the disposable cassette is releasably attached to the housing of the sub-assembly of the reusable drive unit.

Preferably, according to another embodiment, the medicament container of the medicament delivery device is a syringe, a cartridge or a collapsible bag.

Preferably, according to another embodiment, the medicament container of the medicament delivery device is made of glass material or plastic material.

Preferably, according to another embodiment, the medicament container of the medicament delivery device is accommodated within the disposable cassette.

Preferably, according to another embodiment, the medicament delivery device is an injection device, an inhalation device, or a medical sprayer.

Preferably, according to another embodiment, the medicament delivery device is an auto-injector.

Preferably, according to another embodiment, the medicament container is configured to be connected to a medicament delivery member.

Preferably, according to another embodiment, the medicament delivery member is an injection needle or a spray nozzle.

Preferably, according to another embodiment, the medicament delivery member is accommodated in the disposable cassette.

Preferably, according to another embodiment, the medicament delivery device comprises a disposable cassette releasably attached to the housing.

Preferably, according to another embodiment, the disposable cassette is configured to be fully received within the housing of the sub-assembly.

Preferably, according to another embodiment, the disposable cassette is configured to be partially received within the housing of the sub-assembly.

Preferably, according to another embodiment, the disposable cassette comprises a cassette body accommodating the medicament container; a delivery member guard movable relative to the cassette body between an extended position where a delivery member connected to the medicament container is fully surrounded by the delivery member guard and a retracted position where the delivery member is not surrounded by the delivery member guard.

Preferably, according to another embodiment, the information tag is attached to an outer surface of the cassette body.

Preferably, according to another embodiment, the information tag is embedded in the cassette body.

Preferably, according to another embodiment, the information tag is attached to the medicament container.

Preferably, according to another embodiment, when the delivery member guard is moved to the retracted position the delivery member guard is configured to be in contact and move the trigger towards the distal end of the housing such that the sensing area can be detected by the electronic non-contact sensor of the switch.

Preferably, according to another embodiment, the delivery member guard comprises a distally directed surface configured to be engaged with the proximally directed surface of the trigger when the delivery member guard is moved towards the retracted position of the delivery member guard.

Preferably, according to another embodiment, the cassette body comprises a connector and the housing comprises a counter connector configured to be releasably attached to the bayonet connector of the cassette body.

Preferably, according to another embodiment, the cassette body comprises a bayonet connector; wherein the housing comprises a counter bayonet connector configured to be releasably attached to the bayonet connector of the cassette body.

Preferably, according to another embodiment, the cassette body comprises a thread connector; wherein the housing comprises a counter thread connector configured to be releasably attached to the bayonet connector of the cassette body.

Preferably, according to another embodiment, the cassette body comprises a snap-fit connector; wherein the housing comprises a counter snap-fit connector configured to be releasably attached to the bayonet connector of the cassette body.

Preferably, according to another embodiment, a distal part of the cassette is in contact with the second proximally directed surface of the trigger such that when the cassette is rotated around the longitudinal axis relative to the housing, the cassette moves the trigger towards the distal end of the housing.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a medicament delivery device with a sub-assembly of the invention;

FIG. 2-3 schematically show perspective views of components of a housing of the sub-assembly of FIG. 1;

FIG. 4-5 schematically show perspective views of the housing of FIG. 3 a biasing member, and a trigger of the sub-assembly of FIG. 1;

FIG. 6 schematically shows a perspective view of a motor and a lead screw of a sub-assembly of FIG. 1.

FIGS. 7-8 schematically show perspective views of a sensing area and a switch of the sub-assembly of FIG. 1;

FIGS. 9-10 and FIG. 13 schematically show perspective views of the sensing area and the switch of the sub-assembly of FIG. 1;

FIGS. 11-12 schematically show perspective views of a cassette connected to the sub-assembly of FIG. 1;

FIG. 14 schematically shows a perspective view of another cassette that can be connected to the sub-assembly of FIG. 1; and

FIGS. 15-18 schematically show perspective views of the sensing area and the switch of the sub-assembly of FIG. 1 in another example.

### DETAILED DESCRIPTION

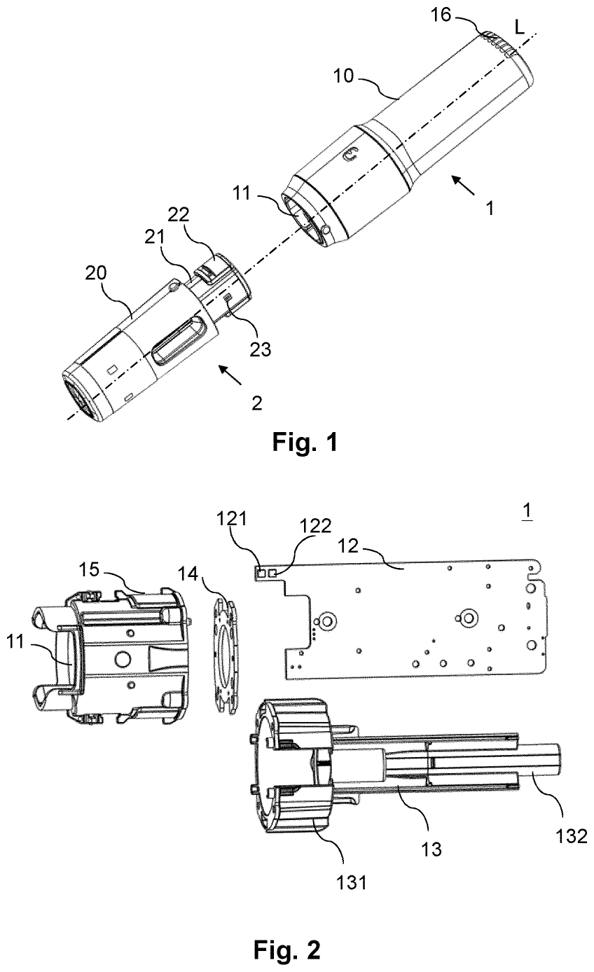

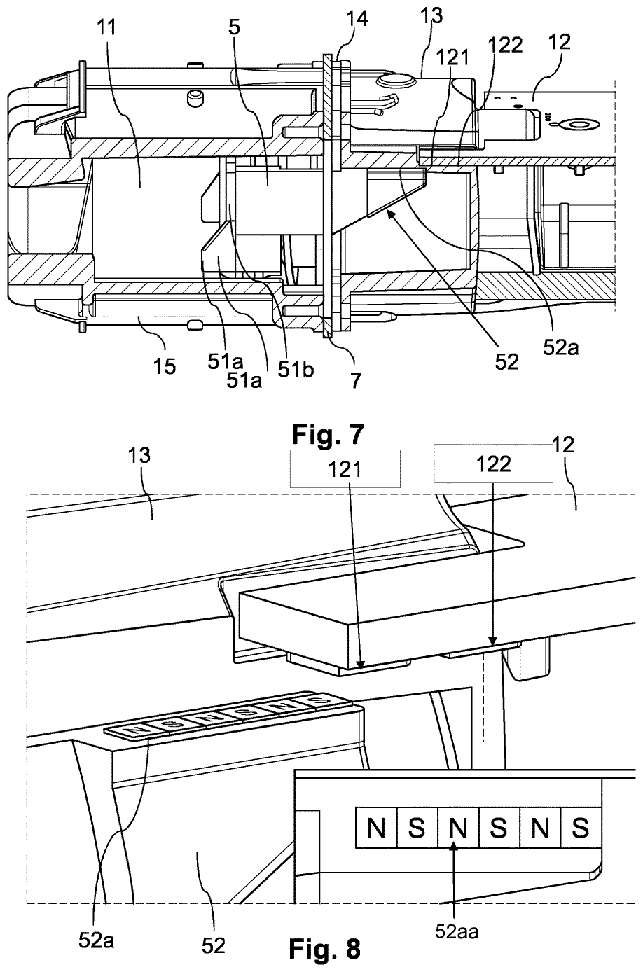

FIGS. 1-18 illustrate a sub-assembly of a medicament delivery device. The sub-assembly can be used in a disposable medicament delivery device or a partially reusable medicament delivery device, namely that a part of the medicament delivery device is disposable, e.g., a medicament delivery member and/or a medicament container, and the rest of parts of the medicament delivery device is reusable, e.g., a motor or other electronic components. The sub-assembly will be explained in detail with the example where the medicament delivery device is a partially reusable medicament delivery device. In this example, the medicament delivery device comprises a reusable drive unit configured to be releasably connected to a disposable cassette 2 comprising a medicament container, as shown in FIG. 1. In this example, the sub-assembly forms a part of the reusable drive unit. Alternatively, the sub-assembly can be used in a disposable medicament delivery device having a built-in medicament container.

In a preferred example, the medicament delivery device is an autoinjector. The medicament container is configured to be connected to a delivery member, e.g., a needle or a spray nozzle. In one example, the medicament container comprises an integral delivery member; for example, the medicament container is a syringe. In another example, a user of the medicament delivery device needs to manually attach a delivery member to the medicament container; for example, the medicament container is a cartridge, and the user needs to attach a pen needle to the cartridge before use. In another example, a delivery member is preassembled to the medicament delivery device but not in fluid communication with the medicament container. A step of establishing fluid communication between the delivery member and the medicament container is carried out before use. For example, the disposable cassette comprises a preassembled delivery member and the medicament container is configured to be moved towards the preassembled delivery member before the medicament delivery operation.

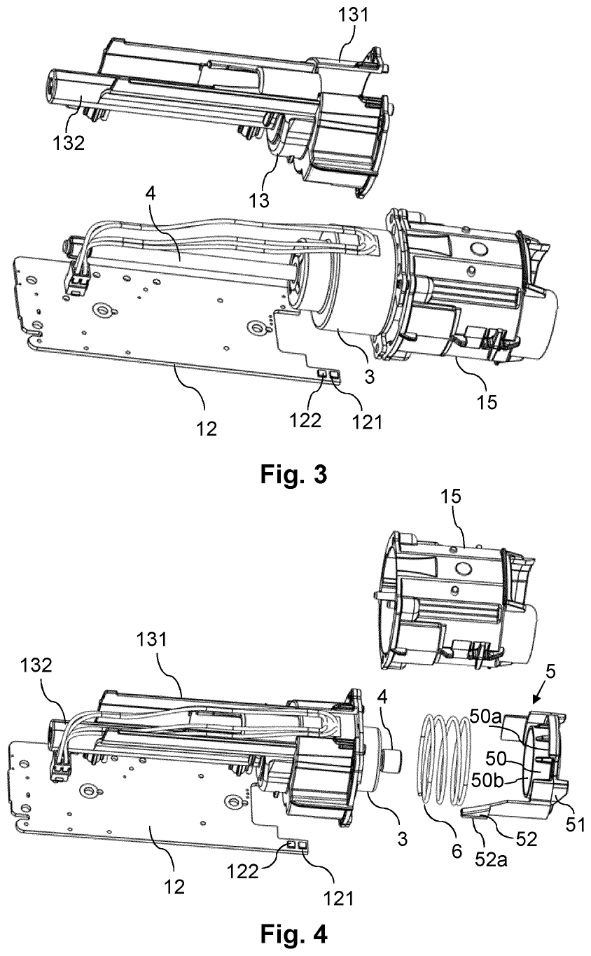

The sub-assembly comprises a housing 1, a processor, a switch, and a trigger 5; 5′; 5″. The housing 1 extends along a longitudinal axis L from a proximal end to a distal end. The housing 1 comprises a distal section and a proximal section. In one example as shown in FIGS. 1-2, the housing optionally comprises an outer shell 10 and an inner shell. In a preferred example, the inner shell is formed by a proximal inner shell 15 and a distal inner shell 13, and optionally, a partition position 14 between the proximal inner shell 15 and the distal inner shell 13, and a printed circuit board (PCB) 12 carrying most of the electronic components (not shown). It should be noted that the housing can be formed in only the outer shell or the inner shell. For example, if the housing only comprises the inner shell, the proximal inner shell 15 is the proximal section of the housing and the distal inner shell 13 is the distal section of the housing. In a preferred example, the PCB is attached to the distal inner shell 13. The partition 14 is attached to both the proximal inner shell 15 and the distal inner shell 13. The proximal inner shell 15 has a channel 11 extending in the direction of the longitudinal axis L. In one example, the housing 1 comprises an indicator 16. Preferably, the indicator 16 is positioned at the distal end of the housing 1. In one example, the indicator 16 is a light indicator, e.g., LED array. The distal inner shell 13 comprises a proximal section 131 and a distal section 132.

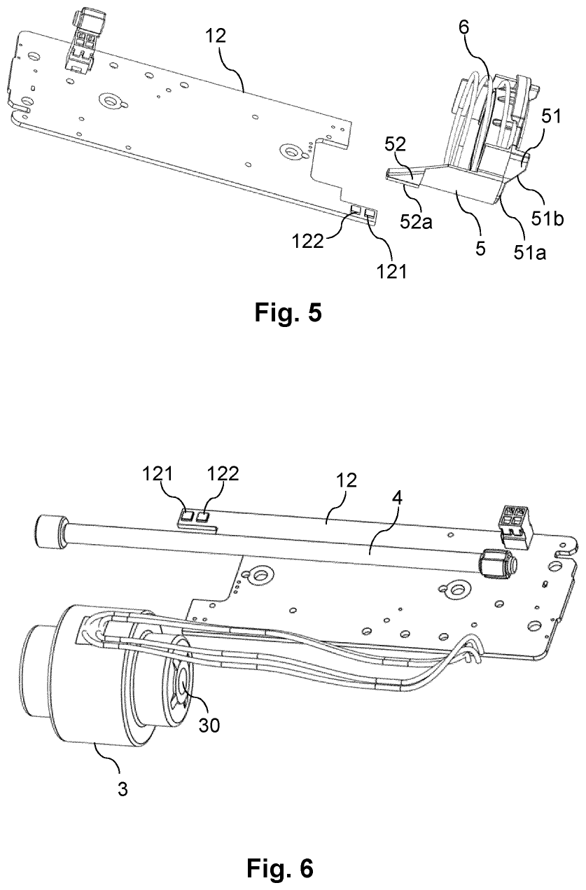

In a preferred example, the sub-assembly comprises a motor 3 accommodated within the housing 1. In a preferred example, the motor 3 is received within the proximal section 131 of the distal inner shell 13, as shown in FIGS. 3-4.

The motor 3 is configured to, upon being activated, cause the expulsion of medicament from the medicament container of the medicament delivery device. In a preferred example, the processor is configured to activate the motor 3; or inactivate the motor 3; or pause the activation of the motor 3.

The motor can be any suitable motor that is capable of outputting force onto the medicament and causing the expulsion of medicament from the medicament container of the medicament delivery device. For example, the motor is configured to output propellant gas towards the medicament. In a preferred example, the motor 3 is configured to drive a lead screw 4 of the sub-assembly to move towards the medicament and thus cause the expulsion of medicament from the medicament container of the medicament delivery device. In this example, the motor 3 can be stepper motor 3. In one example where the sub-assembly comprises the lead screw 4, the lead screw 4 is operably connected to the motor 3 via a thread interface (not shown), preferably, the motor comprises a channel 30 and the lead screw 4 extends through the channel 30 of the motor 3, as shown in FIGS. 3-4, and FIG. 6. In this example, the distal section of the distal inner shell 132 of the housing 1 is configured to prevent the lead screw from rotating around the longitudinal axis L relative to the motor 3. Thus, when the motor 3 is activated, the motor rotates. The rotation of the motor is converted into a linear movement of the lead screw in the direction of the longitudinal axis L due to the thread interface between the lead screw 4 and the motor 3, and the rotation prevention structure between the distal section of the distal inner shell 132 and the lead screw 4. In one example, the lead screw comprises a groove and the distal section of the distal inner shell 132 of the housing comprises a rib positioned within the groove to prevent the rotation of the lead screw relative to the motor.

In one example, the motor is configured to reciprocally move the lead screw in the direction of the longitudinal axis relative to the housing.

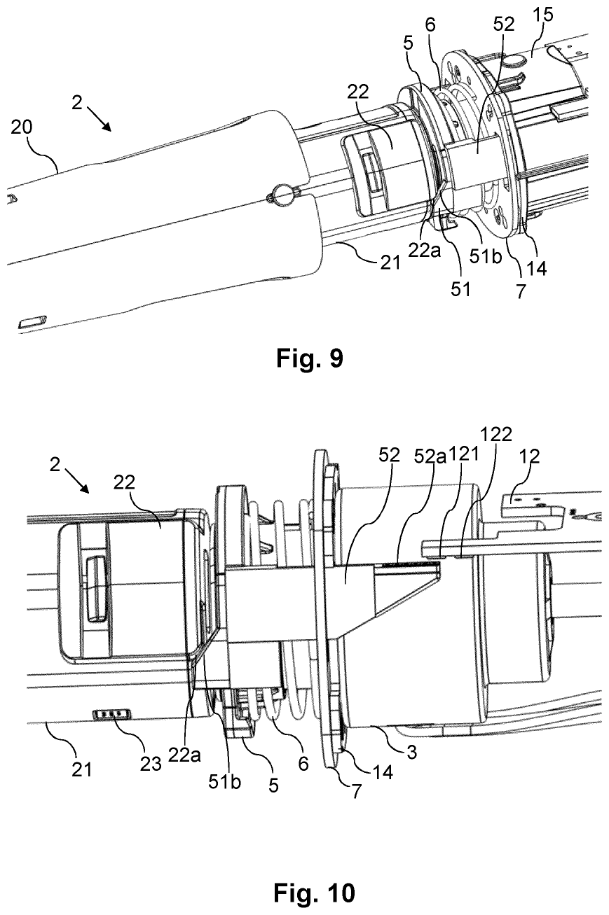

Furthermore, in another example, the sub-assembly comprises a seal 7. The seal 7 is positioned between the distal section of the housing 1 and the proximal section of the housing 1. The switch is fully positioned within the distal section of the housing 1. The trigger 5; 5′; 5″ is positioned within the proximal section of the housing 1, as shown in FIG. 7. In this example, the PCB 12 is positioned within the distal section of the housing. In a preferred example, the seal is made of TPE such that electronic components can be protected within the housing 1 with an ingress protection level 22 (IP22).

The switch is accommodated within the housing 1. The trigger 5; 5′; 5″ is movably accommodated within the housing 1. The trigger 5; 5′; 5″ comprises a sensing area 52a; 52a′; 52a″. The switch comprises an electronic non-contact sensor 121, 122; 121′; 121, 122′ attached to the housing 1 and configured to sense the sensing area 52a; 52a′; 52a″ of the trigger 5; 5′; 5″. In response to sensing the sensing area 52a; 52a′; 52a″, the switch is configured to provide a signal to the processor.

In a preferred example, the processor is configured to, upon received the signal from the switch, activate the motor to expel the medicament contained within the medicament container of the medicament delivery device or inactivate the motor, e.g., switch off the motor completely that the power consumption of the motor can be close to zero (as there is always minor power consumption within electronic component); or pause the activation of the motor, e.g., put the motor to a standby status that the motor can be re-activated quickly.

Alternatively, or additionally, the sub-assembly comprises a communication unit connected to the processor. In this example, the processor, upon received the signal provided by the switch, is configured to activate the communication unit. The communication unit will be explained in detail later.

The trigger 5; 5′; 5″ is configured to be moved by another component of the medicament delivery device within the housing 1 such that the sensing area 52a; 52a′; 52a″ can be moved relative to the electronic non-contact sensor 121, 122; 121′; 121″; 122″ as the electronic non-contact sensor 121, 122; 121′; 121″; 122″ is attached to the housing 1. The component that is configured to move the trigger 5; 5′; 5″ is not a part of the sub-assembly.

In a preferred example, the sensing area 52a; 52a′; 52a″ of the trigger 5; 5′; 5″ comprises a sensing array 52aa having at least two sensing sections respectively lined up in a predetermined sequence. In this example, the electronic non-contact sensor 121, 122; 121′; 121″; 122″ is configured to sense different signals from the at least two sensing sections respectively.

In one example, the sensing area 52a of the trigger 5 comprises a magnet. Alternatively, or additionally, the sensing area of the trigger is made of magnetic materials. Alternatively, or additionally, the sensing array 52aa of the trigger 5 comprises a magnet array having at least two magnets with different polarities in the at least two sensing sections respectively.

In one example, the electronic non-contact sensor is a magnetic sensor configured to provide the signal when the detected a predetermined magnetic field change. In one example, the predetermined magnetic field change is the magnetic field strength is above a predetermined threshold. Alternatively, or additionally, the predetermined magnetic field change is a predetermined sequence of polarity changes. For example, when the sensing array 52aa of the trigger 5 comprises a magnet array having at least two magnets with different polarities in the at least two sensing sections respectively, the electronic non-contact sensor 121; 122 is configured to detect the predetermined sequence of polarity changes from the magnet array. In a preferred example, the magnetic sensor comprises a CMOS process. In another example, the magnetic sensor is a Tunnel Magneto-resistance (TMR) sensor.

For example, the electronic non-contact sensor can comprise a Hall sensor and/or a reed switch and/or a magnetic meter and/or a magnet and/or a coil opening in the direction of the longitudinal axis, the sensor is configured to sense a current change on the coil induced by the change of the magnetic field.

In one example, the processor connected to the electronic non-contact sensor and the motor. Alternatively, or additionally, the switch is a reed switch connected between a motor and a battery.

Furthermore, in another example, the electronic non-contact sensor 121′; 121″; 122″ is an optical sensor. For example, the electronic non-contact sensor can be a photoelectric sensor, a color sensor, an infrared sensor, or a combination thereof.

In one example where the sensing area 52a″ of the trigger 5″ comprises the sensing array having at least two sensing sections respectively lined up in a predetermined sequence, the at least two sensing sections are capable of providing different reflection signal respectively, as shown in FIG. 18. For example, the at least two sensing sections are coated with two different colors respectively. Alternatively, or additionally, the at least two sensing sections are formed in two different structures respectively. In one example, the at least two sensing sections are a concave surface and a convex surface.

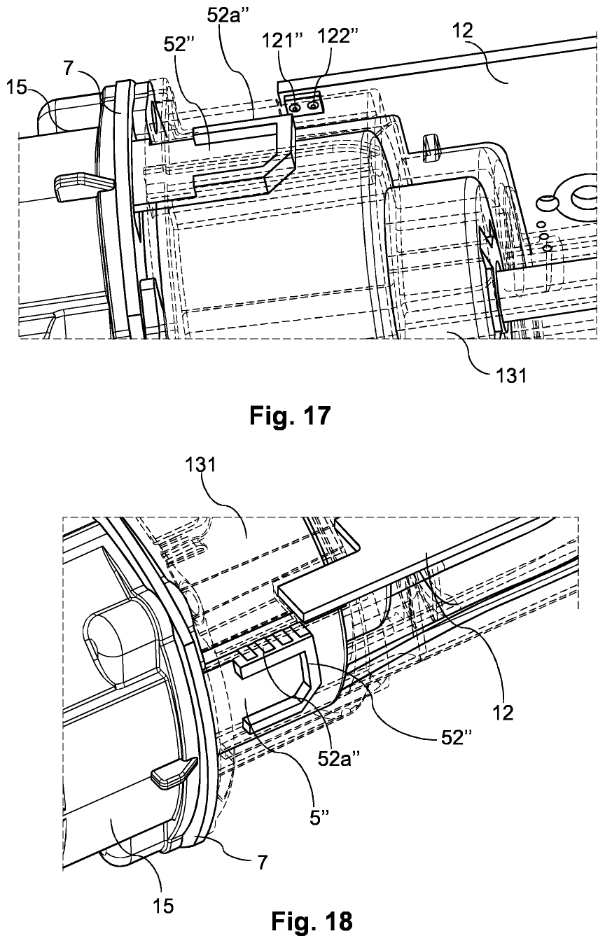

Alternatively, the electronic non-contact sensor 121′ can be an optical mouse sensor being capable of sensing the movement and the moving direction of the sensing area 52a′ of the trigger 5′. In this example, the sensing area 52a′ of the trigger 5′ can a simply a smooth surface facing towards the electronic non-contact sensor 121′, as shown in FIGS. 15-16. Alternatively, or additionally, the electronic non-contact sensor of the switch of the sub-assembly can be an ultrasound sensor configured to detect the appearance and/or the absence of the sensing area. Thus, the sensing area can be made of the same material as the trigger. For example, the sensing area is made of plastic if the trigger is made of plastic.

The trigger 5; 5′; 5″ is movable relative to the housing 1. In one example, the trigger is rotatable relative to the housing around the longitudinal axis. Alternatively, the trigger 5; 5′; 5″ is movable relative to the housing in the direction of the longitudinal axis L. In one example, the trigger 5 comprises a body 50 having a distal section 52; 52′; 52″ and a proximal section 51, as shown in FIG. 4. The distal section 52; 52′; 52″ comprises the sensing area 52a. The proximal section 51 comprises a first proximally directed surface 51a. The first proximally directed surface 51a is configured to be in contact and be moved by another component of the medicament delivery device such that the sensing area 52a can be sensed by the electronic non-contact sensor 121, 122; 121′; 121″, 122″ of the switch. In another example, the proximal section 51 comprises a second proximally directed surface 51b extending from the first proximally directed surface 51a towards the proximal end of the body 50, thus, the second proximally directed surface 51b is a ramp surface, as shown in FIG. 5.

In one example, the body 50 of the trigger 5; 5′; 5″ comprises a distally directed surface 50a and the housing 1 comprises a proximally directed surface (not shown). In this example, the sub-assembly comprises a biasing member 6 positioned between the distally directed surface 50a of the trigger 5; 5′; 5″ and the proximally directed surface of the housing 1 such that the trigger 5; 5′; 5″ is biased towards the proximal end of the housing 1. In this example, when the trigger 5; 5′; 5″ is moved in the distal direction, the sensing area 52a; 52a′; 52a″ can be sensed by the electronic non-contact sensor 121, 122; 121′; 121″, 122″ of the switch, and when the trigger 5; 5′; 5″ is moved back to its original position by the biasing member 6, the sensing area 52a; 52a′; 52a″ can be sensed by the electronic non-contact sensor 121, 122; 121′; 121″, 122″ of the switch again. As a result, different signals can be provided to the processor to control the motor 3. For example, when the trigger 5; 5′; 5″ is moved in the distal direction, the sensing area 52a; 52a′; 52a″ is sensed by the electronic non-contact sensor 121, 122; 121′; 121″, 122″ of the switch, and a signal is provided by the switch to the processor to activate the motor 3, for example, the motor 3 now starts to rotate. As a result, the expulsion of the medicament from a medicament container of the medicament delivery device starts. When the trigger 5; 5′; 5″ is moved back to its original position by the biasing member 6, the sensing area 52a; 52a′; 52a″ can be sensed by the electronic non-contact sensor 121, 122; 121′; 121″, 122″ of the switch again, thus, a signal is provided by the switch to the processor to inactivate the motor 3, for example, the motor 3 now stops to rotate. As a result, the expulsion of the medicament from a medicament container of the medicament delivery device is stopped.

In a preferred example, the biasing member 6 is a spring. Alternatively, the biasing member is a flexible arm extending from the housing 1.

In one example where the sub-assembly comprises the seal 7, an opening is arranged in a wall of the seal 7. In this example, the distal section 51 of the trigger 5; 5′; 5″ comprises the sensing area, and the distal section 51 of the trigger 5; 5′; 5″ is configured to be moved through the opening when the trigger 5; 5′; 5″ is moved towards the distal end of the housing 1. In one example, the distal section 5 of the trigger 5; 5′; 5″ snugly fits the size of the opening of the wall of the seal 7.

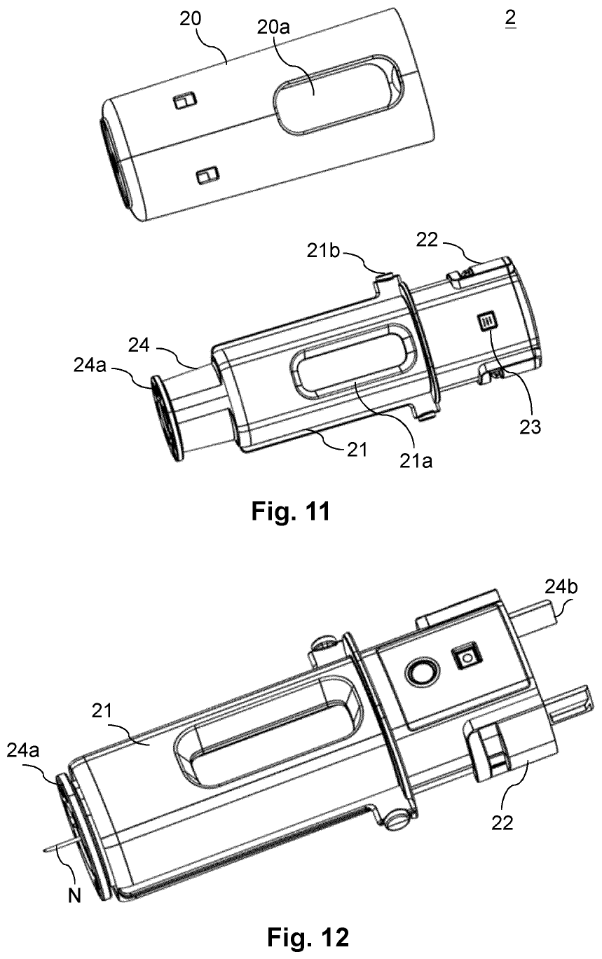

In one example where the sub-assembly comprises the biasing member 6, the biasing member 6 is positioned between the distally directed surface 50a of the trigger 5; 5′; 5″ and a proximally directed surface of the seal 7, as shown in FIG. 9.

In one example where the housing 1 comprises the channel 11, the channel 11 is in the proximal section of the housing 1. The channel 11 is configured to be releasably attached to a disposable cassette 2 of the medicament delivery device. In this example, the proximal section 51 of the body 50 of the trigger 5; 5′; 5″ is positioned within the channel 11 of the housing 1 such that a component of the disposable cassette 2 is capable of being in contact with the proximal section 51 of the body 50 of the trigger 5; 5′; 5″ and moving the trigger 5; 5′; 5″ towards the distal end of the housing 1.

Furthermore, in one example where the sub-assembly comprises the communication unit, preferably, the communication unit is positioned within the channel such that an information tag of the disposable cassette can be read and/or written by the communication unit. In a preferred example, the communication unit is an RFID/NFC reader and/or writer.

In a preferred example, the switch comprises two electronic non-contact sensors lined up to one another in the direction of the longitudinal axis L, as shown in FIGS. 2-9 and FIG. 17. In this example, the switch is configured to provide at least two different signals to the processor based on different combinations of the detections from two electronic non-contact sensors respectively. In one example, the two electronic non-contact sensors are a first sensor 121; 121″ and a second sensor 122; 122″. Different combinations of the detection from the first sensor 121 and the second sensor 122 can be used to provide different signals. One example is shown in Table 1, in this example, the first sensor 121 and the second sensor 122 are magnetic sensor. In a preferred example, the sensing area 52a comprises a magnet array 52aa, as shown in FIG. 8. In this example, more events that are triggered by the movement of the trigger 5 can be detected.

TABLE 1

| Detection from the | Detection from the | Motor controlled |
|---|---|---|
| first sensor 121 | second sensor 122 | by the processor |
| Negative, high | Positive, high | Allow the motor 3 |
| | | to be activated |
| Positive, high | Negative, high | Activate the motor 3 |

A medicament delivery device comprises a reusable drive unit comprising the sub-assembly as mentioned in any examples above is provided. The medicament delivery device comprises a disposable cassette 2 comprising a medicament container containing a medicament. The disposable cassette 2 is releasably attached to the housing 1 of the sub-assembly of the reusable drive unit.

In one example, the disposable cassette 2 comprises a cap 20, a cassette body 21, a connector 22, and an information tag 23, as shown in FIG. 1. The cassette body 21 is configured to accommodate the medicament container. In one example, the cassette body 21 comprises a window 21a such that the medicament container can be observed via the window 21a. In one example, the connector 22 is a bayonet connector configured to be engaged with a counter bayonet connected within the channel 11 of the housing 1. Alternatively, the connector of the cassette can be a threaded connector. Alternatively, the connector of the cassette can be a snap-fit connector.

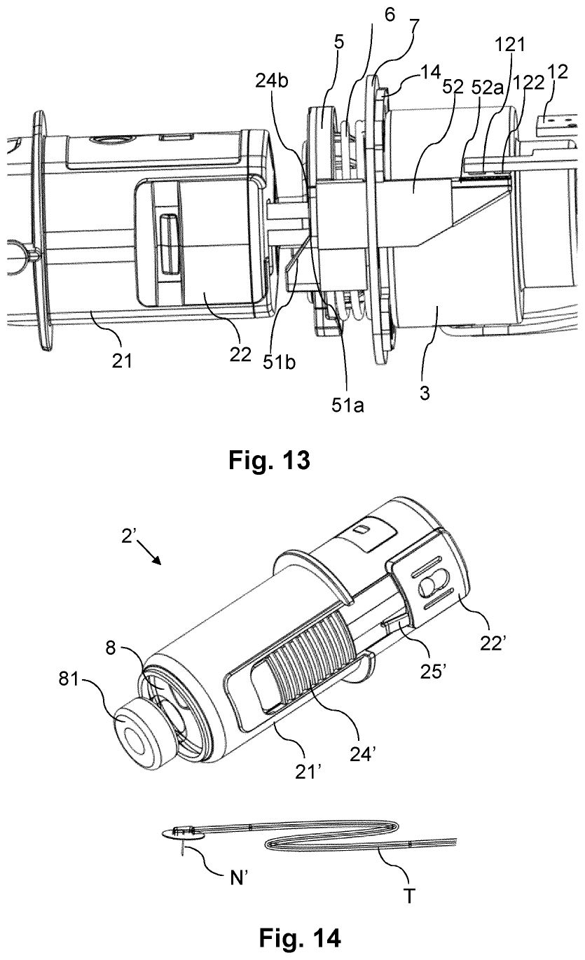

In one example, the disposable cassette 2 comprises a delivery member guard 24 movable relative to the cassette body 21 between an extended position where a delivery member N connected to the medicament container is fully surrounded by the delivery member guard 24, as shown in FIG. 11, and a retracted position where the delivery member N is not surrounded by the delivery member guard 24, as shown in FIG. 12. The delivery member guard 24 comprises a proximal surface 24a configured to be in contact with a medicament delivery site. For initiating the medicament delivery operation, the user needs to press the proximal surface 24a of the delivery member guard against the medicament delivery site such that the delivery member guard 24 is moved in the distal direction relative to the cassette body 21 to the retracted position. The delivery member guard 24 comprises a distally directed surface 24b configured to be engaged with the proximally directed surface of the trigger 5 when the delivery member guard 24 is moved towards the retracted position of the delivery member guard 24. In one example where the trigger 5 comprises the first proximally directed surface 51a, the distally directed surface 24b of the delivery member guard 24 is configured to be engaged with the proximally directed surface of the trigger 5; 5′; 5″ when the delivery member guard 24 is moved towards the retracted position of the delivery member guard 24, as shown in FIG. 13. In one example where the trigger 5; 5′; 5″ comprises the second proximally directed surface 51b and the disposable cassette 2 comprises the bayonet connector 22, the bayonet connector 22 of the disposable cassette 2 comprises a distally directed surface 22a configured to be in contact with the second proximally directed surface 51b of the trigger 5; 5′; 5″, as shown in FIGS. 9-10. In this example, when the disposable cassette 2 is rotated relative to the housing 1 of the sub-assembly around the longitudinal axis L to be attached to the housing 1, the engagement between the distally directed surface 22a of the bayonet connector 22 of the disposable cassette 2 and the second proximally directed surface 51b of the trigger 5; 5′; 5″ converts the rotation of the cassette into an axial movement of the trigger 5 in the distal direction relative to the housing 1.

In one example where the sub-assembly comprises wo electronic non-contact sensors 121, 122 lined up to one another in the direction of the longitudinal axis L, when the disposable cassette 2 is attached to the housing 1, the trigger 5 is moved in the distal direction by the bayonet connector 22; thus, the first sensor 121 senses the sensing area 52b, as shown in FIG. 8. When the delivery member guard 24 is pressed against the medicament delivery site to move to its retracted position, the trigger 5 is further moved by the delivery member guard 24 in the distal direction such that both the first sensor and the second sensor can detect the sensing area 52b, as shown in FIG. 13.

In this example, when the first sensor detects the sensing area 52a; 52a′; 52a″ but the second sensor does not detect the sensing area 52a; 52a′; 52a″, the status of the medicament delivery device can be identified as a pre-activation statue by the processor; when both the first sensor and the second sensor can detect the sensing area 52b, the processor can activate the motor. In this example, the processor is designed not to activate the motor if the medicament delivery device is not in the pre-activation statue. As a result, potential interference from an ambient magnet next to the medicament delivery device can be mitigated. Furthermore, the pre-activating signal can also be used to switch on the communication unit such that when the user attaches the disposable cassette 2 to the housing 1, the communication unit can read the information tag 23 of the disposable cassette 2.

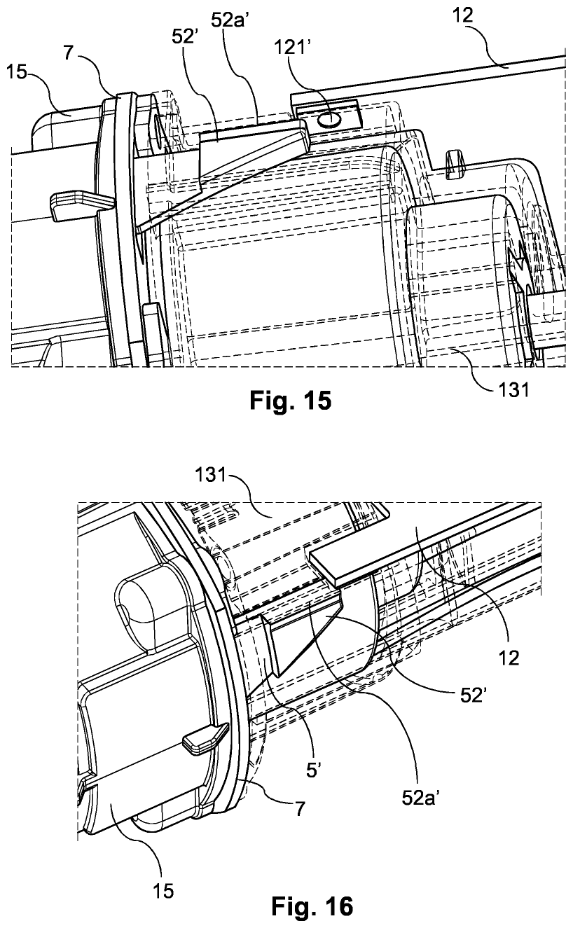

FIG. 14 shows another example of the disposable cassette that can be used with the medicament delivery device as mentioned above. In this example, the disposable cassette does not comprise the delivery member guard as mentioned above. In this example, the disposable cassette 2′ comprises a cassette body 21′, a sliding button 24′, a connector 22′, and optionally, a safety lock 25′. In this example, the disposable cassette 2′ does not comprise a delivery member. In this example, a cartridge 8 is configured to be inserted into the cassette body 21′. In this example, the user is configured to fluidly connect a tubing T coupled to a delivery member N′ to the cartridge outlet 81 before use. In this example, the connector 22′ can be identical to the connector 22 disclosed in the previous example.

In this example, the sliding button 24′ is configured to be moved in the distal direction relative to the cassette body 21′. In this example, the sliding button 24′ is configured to move the trigger 5 in the distal direction relative to the housing 1 such that the sensing area 52b can be sensed by the electronic non-contact sensor 121, 122. In one example the safety lock 25′ is configured to prevent the sliding button 24′ from being moved in the distal direction relative to the cassette body 21′ before the cassette 2′ is attached to the housing 1. For example, the safety lock 25′ is a flexible pin connected to the sliding button 24′ and the cassette body 21′ such that the distal movement of the sliding button 24′ is blocked by the engagement between the flexible pin and the cassette body 21′. In one example where the connector 22′ is the bayonet connector, when the connector 22′ is inserted into the housing 1, a wall of the housing 1 is configured to press the flexible pin 25′ towards the longitudinal axis L such that the flexible pint 25′ is radially offset from the sliding button 24′ and cannot block the distal movement of the sliding button 24′.

As mentioned above, with the two or more electronic non-contact sensors, and/or the sensing array of the sensing area more events that are triggered by the movement of the trigger 5; 5′; 5″ can be detected. Some examples of the operation of the medicament delivery device with the sub-assembly is described below.

In a first example, the sub-assembly is used in reusable drive unit of a medicament delivery device. The medicament delivery device comprises the reusable drive unit and an exchangeable and disposable cassette. The cassette comprises a cassette body receiving a medicament container containing medicament, a needle is attached to the medicament container, a delivery member guard movably arranged within the cassette body, and an information tag placed on the cassette body. In this example, the cassette body comprises a bayonet protrusion at the distal end of the cassette body. The bayonet protrusion is configured to be positioned within a bayonet track of the housing of the sub-assembly and travel along the bayonet track such that the cassette can be attached to the housing of the sub-assembly. In this example, the distal end of the cassette comprises a cover to prevent the medicament container from falling off through the distal end of the cassette body. An opening is in a wall of the cover such that a distal section of the delivery member guard can pass through the opening of the cover and move into the proximal section of the housing of the sub-assembly. In this example, the delivery member guard is configured to be pressed against a medicament delivery site when a user plans to use the medicament delivery device. Once the delivery member guard is pressed against the medicament delivery site, the delivery member guard is moved in the distal direction relative to the cassette body manually by the user of the medicament delivery device. In this example, the motor is connected to a lead screw. The motor is configured to, upon being activated, rotate and thereby cause the lead screw to move in the direction of the longitudinal axis. In this example, the switch comprises the first magnetic sensor and the second magnetic sensor lined up to one another in the direction of the longitudinal axis. The processor connected to the motor and the first and the second electronic magnetic sensors. The sub-assembly further comprises an indicator connected to the processor, a communication unit connected to the processor, and a battery connected to the processor. In this example, the sub-assembly further comprises a dose sensor configured to detect the volume of the medicament that has been expelled. The dose sensor is connected to the processor. The dose sensor can be an encoder to count the rotation of the motor and/or a load cell to detect the reaction force applied on the lead screw and/or an optical emitter and optical receiver to track the position of the proximal end of the lead screw and/or an ultrasonic emitter and ultrasonic receiver to track the position of the proximal end of the lead screw.

In this example, a magnet array is provided on the sensing area. In one example, the magnet array can be arranged as: a south pole adjacent to a north pole, the north pole adjacent to a next south pole, the next south pole adjacent to a next north pole, the next north pole adjacent to a final south pole lined up from a proximal end of the sensing area to a distal end of the sensing area (S-N-S-N-S).

In the first example, when the user attached the cassette to the housing, the distal end of the cassette body is in contact and therefore moves the trigger in the distal direction relative to the housing when the cassette body is inserted into the housing in the distal direction. As mentioned above, the cassette body is configured to attached to the housing via the bayonet connection, meaning that the cassette body will be axially inserted into the bayonet track of the housing, then being rotated around the longitudinal axis relative to the housing. Thus, when the cassette body is axially inserted into the bayonet track, the cassette body moves the trigger in the distal direction relative to the housing; and when the cassette body is rotated around the longitudinal axis relative to the housing, the distal end of the cassette body presses the proximally directed, ramp surface of the trigger, as mentioned above as the second proximally directed surface, thus, the trigger is moved further in the distal direction by the cassette body. In other words, when the disposable cassette is properly attached to the housing, the trigger will be moved two steps in the distal direction relative to the housing.

As a result, the first magnetic sensor senses the south pole of the magnet of the sensing area when the cassette is inserted to the housing but has not been rotated relative to the housing. In response to this detection, the processor switches on the motor, the communication unit, and the indicator. Thus, the indicator provides a light indication to the user to indicate the reusable drive unit is switched on now. Different types of the light indication can be provided regarding the status of the reusable drive unit. For example, if the reusable drive unit is capable of delivering a predetermined dose, a green light indication can be provided; or if the battery is low, a red light can be provided. When the user rotates the cassette body relative to the housing, the first magnetic sensor senses the north pole of the magnet of the sensing area and the second magnetic sensor senses a weak magnetic field from the south pole of the magnet of the sensing area. In response to this detection, the processor activates the communication unit to read the information from the information tag. The processor is now set the delivery program based on the information received from the information tag. When the user presses the delivery member guard against the medicament delivery site, the delivery member guard is moved towards the distal end of the cassette body, as a result, the needle will expose from the delivery member guard, penetrate through the delivery site, and reach the predetermined injection depth under the delivery site. The trigger is moved by the delivery member guard in the distal direction relative to the housing, thus, the processor can be programed that when the first magnetic sensor senses the south pole of the magnet of the sensing area and the second magnetic sensor senses the south pole of the magnet of the sensing area, it means the needle reaches the predetermined injection depth under the delivery site; when the first magnetic sensor senses the north pole of the magnet of the sensing area and the second magnetic sensor senses the north pole of the magnet of the sensing area, it means the needle penetrated the delivery site but not yet reaches the predetermined injection depth under the delivery site. Thus, when the first magnetic sensor senses the south pole of the magnet of the sensing area and the second magnetic sensor senses the south pole of the magnet of the sensing area, the processor activates the motor to cause the expulsion of the medicament from the medicament container; and the processor controls the communication unit such that the communication unit writes the information (the injection is carried out, for example) in the information tag. When the first magnetic sensor senses the north pole of the magnet of the sensing area and the second magnetic sensor senses the north pole of the magnet of the sensing area, the processor controls the indicator to indicate the user that the needle is not in the predetermined injection depth; and the processor controls the communication unit such that the communication unit writes the information in the information tag (the needle is used, for example). When the user lifts the delivery member guard from the medicament delivery site, the delivery member guard is moved in the proximal direction, thus, the trigger is also moved in the proximal direction either by the biasing member or gravity. Thus, the processor pauses the motor when the proximal movement of the sensing area is sensed by the first and the second magnetic sensors. The processor may compare the signal from the dose sensor, if a predetermined dose is delivered, the processor can trigger the motor to move the lead screw back to its original position; alternatively, if the predetermined dose is not delivery, e.g., the user removes the medicament delivery device too early, the processor will not trigger the motor to move the lead screw back to its original position.

In a second example, the sub-assembly is used in reusable drive unit of a medicament delivery device. The medicament delivery device comprises the reusable drive unit and an exchangeable and disposable cassette. The cassette comprises a cassette body receiving a medicament container containing medicament, a sliding button movably arranged within the cassette body, and an information tag placed on the cassette body. In this example, the cassette body comprises a bayonet protrusion at the distal end of the cassette body. The bayonet protrusion is configured to be positioned within a bayonet track of the housing of the sub-assembly and travel along the bayonet track such that the cassette can be attached to the housing of the sub-assembly. In this example, the distal end of the cassette comprises a cover to prevent the medicament container from falling off through the distal end of the cassette body. An opening is in a wall of the cover such that a distal section of the sliding button can be moved through the opening of the cover and move into the proximal section of the housing of the sub-assembly. In this example, the sliding button is configured to be manually slid in the distal direction relative to the cassette body by the user of the medicament delivery device. Once the sliding button is moved in the distal direction relative to the cassette body manually by the user of the medicament delivery device. In this example, the motor is connected to a lead screw. The motor is configured to, upon being activated, rotate and thereby cause the lead screw to move in the direction of the longitudinal axis. In this example, the switch comprises the first magnetic sensor and the second magnetic sensor lined up to one another in the direction of the longitudinal axis. The processor connected to the motor and the first and the second electronic magnetic sensors. The sub-assembly further comprises an indicator connected to the processor, a communication unit connected to the processor, and a battery connected to the processor. In this example, the sub-assembly further comprises a dose sensor configured to detect the volume of the medicament that has been expelled. The dose sensor is connected to the processor. The dose sensor can be an encoder to count the rotation of the motor and/or a load cell to detect the reaction force applied on the lead screw and/or an optical emitter and optical receiver to track the position of the proximal end of the lead screw and/or an ultrasonic emitter and ultrasonic receiver to track the position of the proximal end of the lead screw.

In this example, a magnet array is provided on the sensing area. In one example, the magnet array can be arranged as: a south pole adjacent to a north pole, the north pole adjacent to a next south pole lined up from a proximal end of the sensing area to a distal end of the sensing area (S-N-S).

In the second example, when the user attached the cassette to the housing, the distal end of the cassette body is in contact and therefore moves the trigger in the distal direction relative to the housing when the cassette body is inserted into the housing in the distal direction. As mentioned above, the cassette body is configured to attached to the housing via the bayonet connection, meaning that the cassette body will be axially inserted into the bayonet track of the housing, then being rotated around the longitudinal axis relative to the housing. Thus, when the cassette body is axially inserted into the bayonet track, the cassette body moves the trigger in the distal direction relative to the housing; and when the cassette body is rotated around the longitudinal axis relative to the housing, the distal end of the cassette body presses the proximally directed, ramp surface of the trigger, as mentioned above as the second proximally directed surface, thus, the trigger is moved further in the distal direction by the cassette body. In other words, when the disposable cassette is properly attached to the housing, the trigger will be moved two steps in the distal direction relative to the housing.

As a result, the first magnetic sensor senses the south pole of the magnet of the sensing area when the cassette is inserted to the housing but has not been rotated relative to the housing. In response to this detection, the processor switches on the motor, the communication unit, and the indicator. Thus, the indicator provides a light indication to the user to indicate the reusable drive unit is switched on now. Different types of the light indication can be provided regarding the status of the reusable drive unit. For example, if the reusable drive unit is capable of delivering a predetermined dose, a green light indication can be provided; or if the battery is low, a red light can be provided. When the user rotates the cassette body relative to the housing, the first magnetic sensor senses the north pole of the magnet of the sensing area and the second magnetic sensor senses a weak magnetic field from the south pole of the magnet of the sensing area. In response to this detection, the processor controls the communication unit to read the information from the information tag. The processor is now set the delivery program based on the information received from the information tag. When the user slides the sliding button, the first magnetic sensor senses the north pole of the magnet of the sensing area and the second magnetic sensor senses the south pole of the magnet. In response to this detection, the processor activates the motor to cause the expulsion of the medicament from the medicament container.

When the user releases the sliding button, the sliding button is moved in the proximal direction, thus, the trigger is also moved in the proximal direction either by the biasing member or gravity. Thus, the processor can inactivates/pauses the motor when the proximal movement of the sensing area is sensed by the first and the second magnetic sensors. The processor may compare the signal from the dose sensor, if a predetermined dose is delivered, the processor can trigger the motor to move the lead screw back to its original position; alternatively, if the predetermined dose is not delivery, the processor will not trigger the motor to move the lead screw back to its original position, as in this case, the user might want to continue the medicament delivery operation after a break.

Thus, multiple magnetic sensors and a magnet array arranged of the sensing area can provide more possibility of detections without significantly medicament delivery device cost increasing. Furthermore, in a third example, the sub-assembly is used in reusable drive unit of a medicament delivery device. The medicament delivery device comprises the reusable drive unit and an exchangeable and disposable cassette. The cassette comprises a cassette body receiving a medicament container containing medicament, a needle is attached to the medicament container, a delivery member guard movably arranged within the cassette body, and an information tag placed on the cassette body. In this example, the cassette body comprises a bayonet protrusion at the distal end of the cassette body. The bayonet protrusion is configured to be positioned within a bayonet track of the housing of the sub-assembly and travel along the bayonet track such that the cassette can be attached to the housing of the sub-assembly. In this example, the distal end of the cassette comprises a cover to prevent the medicament container from falling off through the distal end of the cassette body. An opening is in a wall of the cover such that a distal section of the delivery member guard can pass through the opening of the cover and move into the proximal section of the housing of the sub-assembly. In this example, the delivery member guard is configured to be pressed against a medicament delivery site when a user plans to use the medicament delivery device. Once the delivery member guard is pressed against the medicament delivery site, the delivery member guard is moved in the distal direction relative to the cassette body manually by the user of the medicament delivery device. In this example, the motor is connected to a lead screw. The motor is configured to, upon being activated, rotate and thereby cause the lead screw to move in the direction of the longitudinal axis. In this example, the switch comprises the first optical sensor and the second optical sensor lined up to one another in the direction of the longitudinal axis. A processor connected to the motor and the switch. The sub-assembly further comprises an indicator connected to the processor, a communication unit connected to the processor, and a battery connected to the processor. In this example, the sub-assembly further comprises a dose sensor configured to detect the volume of the medicament that has been expelled. The dose sensor is connected to the processor. The dose sensor can be an encoder to count the rotation of the motor and/or a load cell to detect the reaction force applied on the lead screw and/or an optical emitter and optical receiver to track the position of the proximal end of the lead screw and/or an ultrasonic emitter and ultrasonic receiver to track the position of the proximal end of the lead screw.

In this example, the sensing sections of the sensing array are coated with different color respectively. For example, it can be arranged as: a blue section adjacent to a red section, the red section adjacent to a yellow section, the yellow section adjacent to a green section, the green section adjacent to a white section lined up from a proximal end of the sensing area to a distal end of the sensing area (B-R-Y-G-W).

In the third example, when the user attached the cassette to the housing, the distal end of the cassette body is in contact and therefore moves the trigger in the distal direction relative to the housing when the cassette body is inserted into the housing in the distal direction. As mentioned above, the cassette body is configured to attached to the housing via the bayonet connection, meaning that the cassette body will be axially inserted into the bayonet track of the housing, then being rotated around the longitudinal axis relative to the housing. Thus, when the cassette body is axially inserted into the bayonet track, the cassette body moves the trigger in the distal direction relative to the housing; and when the cassette body is rotated around the longitudinal axis relative to the housing, the distal end of the cassette body presses the proximally directed, ramp surface of the trigger, as mentioned above as the second proximally directed surface, thus, the trigger is moved further in the distal direction by the cassette body. In other words, when the disposable cassette is properly attached to the housing, the trigger will be moved two steps in the distal direction relative to the housing.

As a result, the first optical sensor senses the white section of the sensing area when the cassette is inserted to the housing but has not been rotated relative to the housing. In response to this detection, the processor switches on the motor, the communication unit, and the indicator. Thus, the indicator provides a light indication to the user to indicate the reusable drive unit is switched on now. Different types of the light indication can be provided regarding the status of the reusable drive unit. For example, if the reusable drive unit is capable of delivering a predetermined dose, a green light indication can be provided; or if the battery is low, a red light can be provided. When the user rotates the cassette body relative to the housing, the first optical sensor senses the green section of the sensing area and the second optical sensor senses the white section of the sensing area. In response to this detection, the processor activates the communication unit to read the information from the information tag. The processor is now set the delivery program based on the information received from the information tag. When the user presses the delivery member guard against the medicament delivery site, the delivery member guard is moved towards the distal end of the cassette body, as a result, the needle will expose from the delivery member guard, penetrate through the delivery site, and reach the predetermined injection depth under the delivery site. The trigger is moved by the delivery member guard in the distal direction relative to the housing, thus, the processor can be programed that when the first optical sensor senses the yellow section followed by the red and blue sections of the sensing area and the second optical sensor senses the green section followed by the yellow and red sections of the sensing area, it means the needle reaches the predetermined injection depth under the delivery site; when the first optical sensor senses the only the yellow section and/or the red sections of the sensing area and the second optical sensor senses the only the green and/or yellow sections of the sensing area, it means the needle penetrated the delivery site but not yet reaches the predetermined injection depth under the delivery site. Thus, the processor will only activate the motor to cause the expulsion of the medicament from the medicament container; and the processor controls the communication unit such that the communication unit writes the information (the injection is carried out, for example) in the information tag when the needle reaches the predetermined injection depth under the delivery site. When the user lifts the delivery member guard from the medicament delivery site, the delivery member guard is moved in the proximal direction, thus, the trigger is also moved in the proximal direction either by the biasing member or gravity. Thus, the processor pauses the motor when the proximal movement of the sensing area is sensed by the first and the second magnetic sensors. The processor may compare the signal from the dose sensor, if a predetermined dose is delivered, the processor can trigger the motor to move the lead screw back to its original position; alternatively, if the predetermined dose is not delivery, e.g., the user removes the medicament delivery device too early, the processor will not trigger the motor to move the lead screw back to its original position.

The color of the sensing sections as mentioned in the third example can be replaced by different shapes of structures, e.g., protrusions with different shapes, concave-convex; different materials, e.g., different metals, glass, glasses with different thickness; or a combination there of, e.g., a combination of colors and structures.

The delivery member guard in the third example can be replaced by a sliding button as mentioned above in the second example when there is no preassembled needle attached to the medicament container, e.g., the medicament delivery device is an inhalation device or a jet injector, or the user needs to attach a pen needle manually before use.

Furthermore, the housing of the medicament delivery device, as mentioned in any example, may be provided with (i.e., molded in, molded with) a compound featuring persistently antimicrobial, antifungal, and/or antiviral properties. Alternatively, a compound featuring persistently antimicrobial, antifungal, and/or antiviral properties may be applied to the molded (i.e., finished) components through secondary processes (e.g., chemical vapor deposition), spraying, or dipping processes.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of medicaments that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary medicaments that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary medicaments that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary medicaments that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary medicaments that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Exemplary medicaments that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy medicaments include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

The inventive concept has mainly been described above with reference to a few examples. However, as is readily appreciated by a person skilled in the art, other embodiments than the ones disclosed above are equally possible within the scope of the inventive concept, as defined by the appended claims.

Other aspects of the invention are defined by the following clauses.
1. A sub-assembly of a medicament delivery device comprising a medicament container, the sub-assembly comprising:
   a housing (1) extending along a longitudinal axis (L) from a proximal end to a distal end;
   a processor connected to one or more electronic components; wherein the processor is accommodated within the housing (1);
   a switch accommodated within the housing (1);
   a trigger (5) movably accommodated within the housing (1); wherein the trigger (5) comprises a sensing area (52a);
   wherein the switch comprises an electronic non-contact sensor (121, 122) attached to the housing and configured to sense the sensing area (52a) of the trigger (5); and
   wherein in response to sensing the sensing area (52a), the switch is configured to provide a signal to the processor.
2. The sub-assembly according to clause 1, wherein the trigger is relative to the housing in the direction of the longitudinal axis.
3. The sub-assembly according to clause 1 or 2, wherein the sub-assembly comprises a motor accommodated within the housing; wherein the motor is configured to, upon being activated, cause expulsion of a medicament from the medicament container of the medicament delivery device; and wherein the processor, upon received the signal provided by the switch, is configured to activate the motor; or inactivate the motor; or pause the activation of the motor.
4. The sub-assembly according to any one of the preceding clauses, wherein the sub-assembly comprises a communication unit connected to the processor; and wherein the processor, upon received the signal provided by the switch, is configured to activate the communication unit.
5. The sub-assembly according to any one of the preceding clauses, wherein the housing comprises a distal section and a proximal section; wherein the switch is fully positioned within the distal section of the housing; and wherein the trigger is positioned within the proximal section of the housing.
6. The sub-assembly according to clause 5, wherein the proximal section of the housing comprises a channel configured to be releasably attached to a disposable cassette of the medicament delivery device; wherein the medicament container is arranged within the disposable cassette; and wherein a portion of the trigger is positioned within the channel of the housing.
7. The sub-assembly according to clause 6 when dependent on clause 4, wherein the communication unit is positioned within the channel such that upon being activated, the communication unit is capable of reading and/or writing data from/to an information tag on the disposable cassette.
8. The sub-assembly according to any one of the preceding clauses, wherein the sensing area of the trigger comprises a sensing array having at least two sensing sections respectively lined up in a predetermined sequence; and wherein the electronic non-contact sensor is configured to sense different signals from the at least two sensing sections respectively.
9. The sub-assembly according to any one of the preceding clauses, wherein the sensing area of the trigger comprises a magnetic element.
10. The sub-assembly according to a combination of clause 8 and clause 9, wherein the sensing array of the trigger comprises a magnet array having at least two magnets with different polarities.
11. The sub-assembly according to any one of the preceding clauses when dependent on clause 9, wherein the electronic non-contact sensor is a magnetic sensor; and wherein the switch is configured to provide the signal when the detected a predetermined magnetic field change.
12. The sub-assembly according to clause 11, wherein the predetermined magnetic field change can be at least one of the magnetic field strength exceeding a predetermined threshold, or a predetermined sequence of polarity changes.
13. The sub-assembly according to any one of the preceding clauses, wherein the electronic non-contact sensor comprises a Hall sensor.
14. The sub-assembly according to clause any one of clauses 1-13, wherein the electronic non-contact sensor comprises a coil opening in the direction of the longitudinal axis.
15. The sub-assembly according to clause any one of clauses 1-13, wherein the electronic non-contact sensor is an optical sensor.
16. The sub-assembly according to clause 15, wherein the electronic non-contact sensor is a photoelectronic sensor, a color sensor, or a combination thereof.
17. The sub-assembly according to a combination of clause 8 and any one of clauses 15-16, wherein the at least two sensing sections are capable of providing different reflection signal respectively.
18. The sub-assembly according to clause 17, wherein the at least two sensing sections are coated with two different colors respectively.
19. The sub-assembly according to clause 17, wherein the at least two sensing sections are formed in two different structures respectively.
20. The sub-assembly according to clause 19, wherein the at least two sensing sections are a concave surface and a convex surface.
21. The sub-assembly according to any one of the preceding clauses, wherein the switch comprises two electronic non-contact sensors lined up to one another in the direction of the longitudinal axis.
22. The sub-assembly according to clause 21, wherein the switch is configured to provide at least two different signals to the processor based on different combinations of the detections from two electronic non-contact sensors respectively.
23. The sub-assembly according to any one of the preceding clauses, wherein the trigger comprises a body having a distal section and a proximal section; wherein the distal section comprises the sensing area.
24. The sub-assembly according to clause 23, wherein the proximal section comprises a first proximally directed surface and a second proximally directed surface extending from the first proximally directed surface towards a proximal end of the body.
25. The sub-assembly according to clause 23 or 24, wherein the body of the trigger comprises a distally directed surface; wherein the housing comprises a proximally directed surface; and wherein the sub-assembly comprises a biasing member positioned between the distally directed surface of the trigger and the proximally directed surface of the housing such that the trigger is biased towards the proximal end of the housing.
26. A medicament delivery device comprising a reusable drive unit comprising the sub-assembly according to any one of the preceding clauses; wherein the medicament delivery device comprises a disposable cassette comprising a medicament container containing medicament; wherein the disposable cassette is releasably attached to the housing of the sub-assembly of the reusable drive unit.
27. The medicament delivery device according to clause 26, wherein the disposable cassette comprises a cassette body accommodating the medicament container; a delivery member guard movable relative to the cassette body between an extended position where a delivery member connected to the medicament container is fully surrounded by the delivery member guard and a retracted position where the delivery member is not surrounded by the delivery member guard.
28. The medicament delivery device according to clause 26, wherein when the delivery member guard is moved to the retracted position the delivery member guard is configured to be in contact and move the trigger towards the distal end of the housing such that the sensing area can be detected by the electronic non-contact sensor of the switch.
29. The medicament delivery device according to clause 26 when dependent on clause 21, wherein the delivery member guard comprises a distally directed surface configured to be engaged with the proximally directed surface of the trigger when the delivery member guard is moved towards the retracted position of the delivery member guard.
30. The medicament delivery device according to any one of clauses 25-27, wherein the cassette body comprises a bayonet connector; wherein the housing comprises a counter bayonet connector configured to be releasably attached to the bayonet connector of the cassette body.
31. The medicament delivery device according to clause 27 when dependent on clause 21; wherein a distal part of the cassette is in contact with the second proximally directed surface of the trigger such that when the cassette is rotated around the longitudinal axis relative to the housing, the cassette moves the trigger towards the distal end of the housing.

1-15. (canceled)

16. A sub-assembly of a medicament delivery device comprising a medicament container, the sub-assembly comprising:
a housing extending along a longitudinal axis from a proximal end to a distal end, wherein the housing is configured to be attached to a disposable cassette of the medicament delivery device;
a processor connected to one or more electronic components, wherein the processor is accommodated within the housing;
a switch accommodated within the housing; and
a trigger movably accommodated within the housing, wherein the trigger comprises a sensing area, and wherein the trigger is configured to be move by a part of the disposable cassette,
wherein the switch comprises an electronic non-contact sensor attached to the housing and configured to sense the sensing area of the trigger, and
wherein in response to sensing the sensing area, the switch is configured to provide a signal to the processor.

17. The sub-assembly according to claim 16, wherein the trigger is relative to the housing in the direction of the longitudinal axis.

18. The sub-assembly according to claim 16, wherein the sub-assembly comprises a motor accommodated within the housing, wherein the motor is configured to, upon being activated, cause expulsion of a medicament from the medicament container of the medicament delivery device, and wherein the processor, upon received the signal provided by the switch, is configured to (i) activate the motor, (ii) inactivate the motor, or (iii) pause the activation of the motor.

19. The sub-assembly according to claim 16, wherein the sub-assembly comprises a communication unit connected to the processor, and wherein the processor, upon received the signal provided by the switch, is configured to activate the communication unit.

20. The sub-assembly according to claim 16, wherein the housing comprises a distal section and a proximal section.

21. The sub-assembly according to claim 20, wherein the switch is fully positioned within the distal section of the housing, and wherein the trigger is positioned within the proximal section of the housing.

22. The sub-assembly according to claim 20, wherein the proximal section of the housing comprises a channel configured to be releasably attached to a disposable cassette of the medicament delivery device, wherein the medicament container is arranged within the disposable cassette, and wherein a portion of the trigger is positioned within the channel of the housing.

23. The sub-assembly according to claim 22, wherein the communication unit is positioned within the channel such that upon being activated, the communication unit is capable of reading and/or writing data from/to an information tag on the disposable cassette.

24. The sub-assembly according to claim 16, wherein the sensing area of the trigger comprises a sensing array having at least two sensing sections respectively lined up in a predetermined sequence, and wherein the electronic non-contact sensor is configured to sense different signals from the at least two sensing sections respectively.

25. The sub-assembly according to claim 16, wherein the sensing area of the trigger comprises a magnetic element, and wherein the sensing array of the trigger comprises a magnet array having at least two magnets with different polarities.

26. The sub-assembly according to claim 25, wherein the electronic non-contact sensor is a magnetic sensor, and wherein the switch is configured to provide the signal when the detected a predetermined magnetic field change.

27. The sub-assembly according to claim 16, wherein the electronic non-contact sensor is an optical sensor.

28. The sub-assembly according to claim 24 wherein the at least two sensing sections are capable of providing different reflection signal respectively.

29. The sub-assembly according to claim 16, wherein the switch comprises two electronic non-contact sensors lined up to one another in the direction of the longitudinal axis.

30. The sub-assembly according to claim 29, wherein the switch is configured to provide at least two different signals to the processor based on different combinations of the detections from two electronic non-contact sensors respectively.
